(19) United States
(12) Patent Application Publication
(10) Pub. No.: US 2026/0284426 A1
(43) Pub. Date: Sep. 24, 2026

(54) PHOTOIMMUNOTHERAPY AGAINST EXUDATIVE AGE-RELATED MACULAR DEGENERATION

(71) Applicant: Tsubota Laboratory, Inc., Shinjuku-ku, Tokyo (JP)

(72) Inventors: Norimitsu BAN, Tokyo (JP); Toshihide KURIHARA, Tokyo (JP); Hideto OSADA, Tokyo (JP)

(21) Appl. No.: 19/473,416

(22) PCT Filed: Apr. 11, 2024
(86) PCT No.: PCT/JP2024/014636

§ 371 Date: Oct. 7, 2025

(30) Foreign Application Priority Data
Apr. 11, 2023 (JP) 2023-064430

(51) Int. Cl. A61N 5/06; A61K 39/00; A61K 41/00; A61K 47/68; A61P 27/02

(52) U.S. Cl. CPC A61N 5/062; A61K 41/0071; A61K 47/6849; A61P 27/02; A61K 2039/505; A61N 2005/0663

(57) ABSTRACT

A purpose of the present invention is to provide a novel therapeutic strategy for exudative age-related macular degeneration. It was discovered that choroidal neovascularization can be regressed by targeting a VEGF receptor 2 (VEGFR2) which is highly expressing in a choroidal neovascular, for treatment. Therefore, a therapeutic strategy for exudative age-related macular degeneration which includes administering a conjugate of an anti-VEGFR2 antibody and a photosensitizer to a subject, and irradiating red visible light rays or near-infrared light rays to an eye of the subject is provided.

### TECHNICAL FIELD

The present invention relates to photoimmunotherapy for exudative age-related macular degeneration.

### BACKGROUND ART

Age-related macular degeneration (AMD) is a disease in which abnormal symptoms of aging phenomenon occur in a site called as macula of a central part of retina, and a visual function (visual power, visual field) is declined. The age-related macular degeneration is of two types namely, ‘atrophic age-related macular degeneration’ and ‘exudative age-related macular degeneration’. The atrophic age-related macular degeneration is a phenomenon in which macular tissue undergoes atrophic degeneration with aging. Whereas, in the exudative age-related macular degeneration, a neovascular vessel is developed in choroid coat under macula, and grows toward retina. The neovascular vessel is an abnormal blood vessel which does not exist in a normal retina, and a blood vessel wall being extremely weak, leakage of blood component and bleeding are susceptible to occur, and these exudates impair a macular function and cause vision disorder.

As a therapy for the exudative age-related macular degeneration, photodynamic therapy (PDT) in which, laser light is irradiated to a lesion after administering a photosensitizer (VISUDYNE) into the body, is a treatment covered by insurance since 2004 in Japan. Moreover, an anti-VEGF (Vascular Endothelial Growth Factor) antibody (antiangiogenic drug) which inhibits the growth of neovascular vessel was approved in 2008 and 2009. Furthermore, an antibody therapy for a VEGF receptor which has also been proposed (Non-Patent document 1) has shown effectiveness in experiments on animals but has not been applied in a clinical setting.

On the other hand, photoimmunotherapy is a therapeutic strategy in which a conjugate of a photosensitizer and an antibody attached to a lesion site is used, and specifically, it is a therapy of damaging a lesional tissue by changing physical properties by generation of fluorescent light and heat by IR700 by irradiating to lesion red visible light rays or near-infrared light rays that are harmless to living body upon systemic administration of an antibody IR700 conjugate in which, IR700 which is a fluorescent probe is attached to a molecular target monoclonal antibody (Non-Patent document 2). Regarding the photoimmunotherapy, the current research related to cancer treatment has been carried out actively (Patent Documents 1, 2), and in Japan, cetuximab sarotalocan sodium (applicable to cancer of head and neck) was approved in September, 2020.

Moreover, in Patent Document 2, there is a description of an invention related to a conjugate of an antibody targeting a blood vessel and a photosensitizer, in which it is described that a damage by photoimmunotherapy may be caused to a degenerated site of macula that has undergone age-related macular degeneration due to choroidal neovascularization, similarly as to a tumor. However, Patent Document 2 has only disclosed examples in which a decrease in intratumoral neovascularization was observed, without disclosing any result demonstrating the decrease in choroidal neovascularization, and even in Patent Document 2, it was not clear whether or not the invention related to a conjugate of an antibody targeting a blood vessel and a photosensitizer can be used for the treatment of age-relate macular degeneration. In other words, even if the invention is such that a person skilled in the art is able to understand from the content described in a publication or from the content equivalent to that described in the publication, an invention of a method about which it is unclear that the person skilled in the art can use that method based on the description in the publication and technical common sense, it cannot be cited as a cited invention (examination standards), and the invention which can be understood from the afore-mentioned description in Patent Document 2, is merely a wishful thinking without any substantiating experimental results, and falls under the category of inventions for which it is unclear that it can be used, therefore it could not be cited as a cited invention. Moreover, in the examples in which the decrease in intratumoral neovascularization was observed in Patent Document 2, an exposure irradiation dose of the red visible light rays or the near-infrared light rays was 100 J/cm², and the burden on the subject was heavy.

### CITATION LIST

### Patent Documents

Patent Document 1: U.S. Pat. No. 8,524,239 Specification
Patent Document 2: International Patent No. WO2019/172110

### Non-Patent Documents

Non-Patent Document 1: Huang H et al. VEGF receptor blockade markedly reduces retinal microglia/macrophage infiltration into laser-induced CNV.PLoS One. 2013
Non-Patent Document 2: Mitsunaga M et al Cancer cell-selective in vivo near infrared photoimmunotherapy targeting specific membrane molecules. Nat Med. 2011

### SUMMARY OF THE INVENTION

### Problems to be Solved by the Invention

Therapeutic efficacy of photodynamic therapy as a therapeutic strategy for exudative age-related macular degeneration being limited, a treatment by injecting an anti-VEGF antibody intravitreally is predominant currently, and not only that there are examples of resistance to the therapy, but even for the cases of successful treatment, there are many cases in which, generally administration over a long period of time is necessary. The reason being that, although it is possible to reduce the choroidal neovascularization which is an essential pathological condition in the anti-VEGF antibody therapy, the curative regression thereof is not possible. For this reason, establishing a more curative novel therapeutic strategy for exudative age-related macular degeneration is a matter of urgency.

Moreover, in an example in which the reduction in intratumoral neovascularization was observed in Patent Document 2, an exposure irradiation dose of red visible light rays or near-infrared light rays is 100 J/cm² which leads to a heavy burden on the subject, and a method which can treat exudative age-related macular degeneration even with less burden on the subject is sought.

### Means for Solving the Problems

Inventors of the present invention discovered that choroidal neovascularization can be regressed by targeting a VEGF receptor 2 (VEGFR2) which is highly expressing in choroidal neovascularization even with relatively lesser exposure irradiation dose of the red visible light rays or the near-infrared visible light rays and lesser burden on a subject. The present invention, based on such findings, provides a more curative novel therapeutic strategy for exudative age-related macular degeneration, and includes the following aspects.

### [Aspect 1]

A method for treating exudative age-related macular degeneration for a subject requiring treatment, includes administering a conjugate of an anti-VEGFR2 antibody and a photosensitizer to the subject, and irradiating red visible light rays or near-infrared light rays to an eye of the subject.

### [Aspect 2]

The method according to aspect 1, wherein the conjugate of the anti-VEGFR2 antibody and the photosensitizer is administered intravitreally.

### [Aspect 3]

The method according to aspect 1, wherein the conjugate of the anti-VEGFR2 antibody and the photosensitizer is administered intravenously.

### [Aspect 4]

The method according to any one of aspects 1 to 3, wherein a wavelength of the red visible light rays or the near-infrared light rays is in a range of 650 nm to 900 nm.

### [Aspect 5]

The method according to aspect 1, wherein an exposure irradiation dose of the red visible light rays or the near-infrared light rays is in a range of 1 J/cm² to 75 J/cm².

### [Aspect 6]

The method according to aspect 1, wherein an irradiation output of the red visible light rays or the near-infrared light rays is in a range of 100 mW/cm² to 1000 mW/cm².

### [Aspect 7]

The method according to aspect 1, wherein an irradiation time of the red visible light rays or the near-infrared light rays is in a range of 40 seconds to 180 seconds.

### [Aspect 8]

The method according to any one of aspects 1 to 4, wherein the photosensitizer is IR700.

### [Aspect 9]

The method according to any one of aspects 1 to 5, wherein irradiating the red visible light rays or the near-infrared light rays to an eye of the subject includes irradiating the red visible light rays or the near-infrared light rays by a laser irradiating apparatus while observing a lesion site by using a slit-lamp microscope.

### [Aspect 10]

The method according to aspect 1, wherein irradiating the red visible light rays or the near-infrared light rays to the eye of the subject is carried out after a time in a range of 10 minutes to 168 hours after administering the conjugate of the anti-VEGFR2 antibody and the photosensitizer to the subject.

### [Aspect 11]

A pharmaceutical composition for treating exudative age-related macular degeneration includes a therapeutically effective amount of a conjugate of an anti-VEGFR2 antibody and a photosensitizer.

### [Aspect 12]

A use of a therapeutically effective amount of a conjugate of an anti-VEGFR2 antibody and a photosensitizer is for manufacturing a pharmaceutical composition for treating exudative age-related macular degeneration.

### Effect of the Invention

According to the present invention, it is possible to provide a method which can treat exudative age-related macular degeneration even with a less burden on the subject.

### BRIEF DESCRIPTION OF THE DRAWINGS

FIG. 1 is a graph indicating a volume of choroidal neovascularization in exudative age-related macular degeneration model mice in an example;

FIG. 2 is a graph indicating a volume of choroidal neovascularization in exudative age-related macular degeneration model mice in an example in which a spot laser for a PDT was used;

FIG. 3 is a graph indicating a volume of choroidal neovascularization in exudative age-related macular degeneration model mice in an example having administered intravitreally; and

FIG. 4 are photographs indicating a choroidal flat-mount 24 hours after administering a DC101-Alexa488 conjugate.

### MODE FOR CARRYING OUT THE INVENTION

One aspect of the present disclosure is related to a method for treating age-related macular degeneration in which a conjugate of an anti-VEGFR2 antibody and a photosensitizer is used. When the inventors of the present invention studied the therapeutic efficacy of VEGFR2-targeted photoimmunotherapy for age-related macular degeneration by focusing attention on VEGF receptor 2 (VEGFR2) which is highly expressing in choroidal neovascularization of the age-related macular degeneration, they discovered that the choroidal neovascularization which is an essential of the age-related macular degeneration can be regressed by the conjugate of the anti-VEGFR2 antibody and the photosensitizer of the present invention.

### <Conjugate of Anti-VEGFR2 Antibody and Photosensitizer>

### Antibody

In the present invention, as an antibody, a vascular endothelial growth factor (VEGF) receptor 2 (VEGFR2)-targeted antibody which is highly expressing in choroidal neovascularization, preferably a monoclonal antibody, is used.

The anti-VEGFR2 antibody in the present invention may be any one of IgM, IgD, IgG, IgA, and IgE, or may be a chimeric antibody, a humanized antibody, or a fully human antibody, or may be a hybridoma antibody or a recombinant antibody.

Moreover, the anti-VEGFR2 antibody in the present invention may be a full length of immunoglobulin (or a variant thereof) or a partial fragment of immunoglobulin. The partial fragment may be a Fab fragment, a Fab′ fragment, F(ab)′ fragment, a single chain Fv protein (scFv) or a disulfide-stabilized Fv protein (dsFv).

In one preferable aspect, the anti-VEGFR2 antibody is ramucirumab or DC101. In one preferable aspect, the anti-VEGFR2 antibody is an antibody which includes a CDR same as a CDR in ramucirumab or DC101, an antibody which includes a light-chain and a heavy-chain variable region sequence same as a light-chain and a heavy-chain variable region sequence in ramucirumab or DC101, or an antibody which includes a light-chain and a heavy-chain sequence same as a light-chain and a heavy-chain sequence in ramucirumab or DC101.

In the present invention, the anti-VEGFR2 antibody has a function of delivering the photosensitizer to a neovascularization lesion, causing regression of the neovascularization by a light-amplifying effect of the photosensitizer delivered. Moreover, it is preferable that the anti-VEGFR2 antibody also has a function of antagonizing attachment of VEGF and VEGFR2 in addition to the function of delivering the photosensitizer to neovascularization lesion.

### Photosensitizer

The photosensitizer in the present invention has an absorption wavelength region which overlaps with a wavelength region of the red visible light rays or the near-infrared light rays that will be described later. The photosensitizer delivered to the lesion by the anti-VEGFR2 antibody generates heat and an extremely small amount of activated oxygen by irradiation of the red visible light rays or the near-infrared light rays, and can cause damage to neovascularization lesions.

The photosensitizer is not restricted and examples thereof include porphyrin pigments, phthalocyanine naphthalocyanine pigments having the absorption wavelength region overlapping with the wavelength region of the red visible light rays or the near-infrared light rays.

One example of a preferable photosensitizer is IRDye (registered trademark) 700DX(IR700) which is denoted by the following formula provided by LI-COR Biosciences, or a derivative thereof.

[Chemical formula 1]

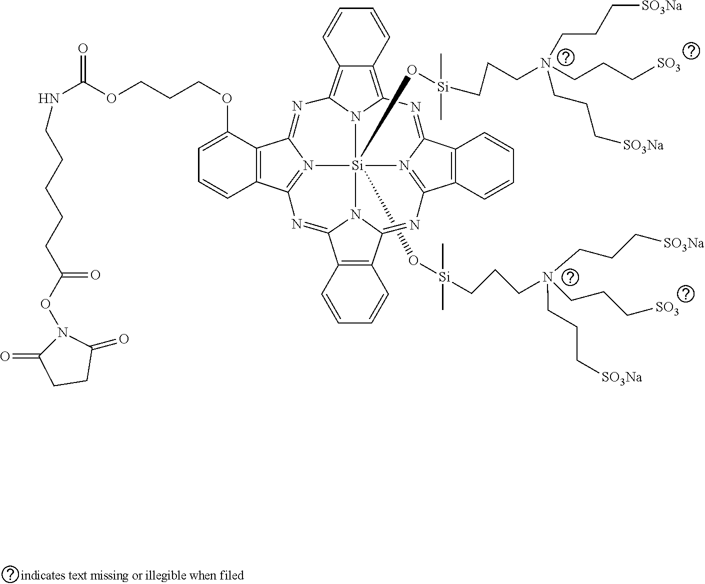

Other examples include various chemical compounds (such as IRDye (registered trademark) 800CW, IRDye (registered trademark) 680RD, IRDye (registered trademark) 680 LT) included in IRDye (registered trademark) Infrared Dyes provided by the same LI-COR Biosciences. Moreover, the photosensitizer may be a porphyrin derivative used in a photodynamic therapy (PDT). The photosensitizer may be a fluorescent material or a light-emitting material.

An attachment of the anti-VEGFR2 antibody and the photosensitizer may be a covalent attachment or a noncovalent attachment. The photosensitizer may be attached directly to the antibody or may be attached to the antibody via a linker or an arbitrary antibody binding molecule.

In one embodiment, it is possible to prepare the conjugate of the anti-VEGFR2 antibody and the photosensitizer easily by using a photosensitizer having an NHS ester group which reacts with an amino group of the antibody (for example, an amino group located in a constant region of the antibody) or a maleimide group which reacts with a thiol group of the antibody.

In one preferable aspect, the conjugate of the anti-VEGFR2 antibody and the photosensitizer is ramucirumab sarotalocan sodium which consists of tetrasodium salt of sarotalocan (6-({[3-({(OC-6-13)-bis({3-[bis(3-sulfopropyl)(3-sulfonatopropyl)azaniumyl]propyl}dimethylsilanolato-κO, κO′) [(phthalocyaninato(2-)κN29, κN30, κN31, κN32)-1-yl]silicon}oxy)propoxy]carbonyl}amino) hexanoyl (C₇₀H₉₆N₁₁O₂S₆S₁₃; molecular weight: 1,752.22)) attached to an average of two to three Lys residues of ramucirumab.

### <Administration of the Conjugate of the Anti-VEGFR2 Antibody and Photosensitizer>

The conjugate of the anti-VEGFR2 antibody and the photosensitizer can be administered by an arbitrary method of administration in which the conjugate finally reaches a retinal lesion. The conjugate of the anti-VEGFR2 antibody and the photosensitizer may be administered as an arbitrary pharmaceutical composition which includes the conjugate. Therefore, one aspect of the present invention is related to a pharmaceutical composition which includes a therapeutically effective amount of the conjugate of the anti-VEGFR2 antibody and the photosensitizer, and particularly a pharmaceutical composition which includes a therapeutically effective amount of the conjugate of the anti-VEGFR2 antibody and the photosensitizer for the treatment of age-related macular degeneration. Moreover, one aspect of the present invention is related to a use of a therapeutically effective amount of a conjugate of the anti-VEGFR2 antibody and the photosensitizer for manufacturing a pharmaceutical composition for treating the age-related macular degeneration.

In the present specification, the conjugate of the anti-VEGFR2 antibody and the photosensitizer and the pharmaceutical composition of the conjugate of the present invention are sometimes collectively referred to as the pharmaceutical composition including the conjugate of the anti-VEGFR2 antibody and the photosensitizer.

In one embodiment, the pharmaceutical composition including the conjugate of the anti-VEGFR2 antibody and the photosensitizer can be administered by topical ocular administration, such as intraocular administration. An intraocular administration route includes administering intravitreally, intraretinally, subretinally, periorbitally, retro orbitally, corneally, and sclerally. Generally, an ocular penetration of an antibody being poor due to the existence of blood-retina barrier, the intravitreal administration is more preferable.

Other examples of topical administration to an eye include eye drop administration (including eye ointment instillation and eye washing), subconjunctival administration, intraconjunctival administration, and subtenon administration.

In one embodiment, the pharmaceutical composition of the conjugate of the anti-VEGFR2 antibody and the photosensitizer can be administered by an arbitrary administration route other than the topical ocular administration, such as enteral administration (orally, via tube, by injecting etc.), parenteral administration (by injecting intravenously, arterially, dermally, or intramuscularly). In general, an antibody pharmaceutical drug is decomposed by digestive enzymes and cannot pass easily through a cell membrane and enter into the cell, thereby sometimes making it difficult to adopt oral administration. Therefore, in one embodiment, the intravenous administration which enables to circulate directly in the whole body is more preferable.

A frequency of administration of the pharmaceutical composition which includes the conjugate of the anti-VEGFR2 antibody and the photosensitizer may be a single dose or multiple doses. In one embodiment, it is a single dose. Even in the previous report (Non-Patent document 1), a contraction of choroidal neovascular by the anti-VEGFR2 antibody has been reported, and multiple doses were necessary. Whereas, in the method for treating by using the conjugate of the anti-VEGFR2 antibody and the photosensitizer of the present invention, the photoimmunotherapy is applied, and by causing damage directly to the choroidal neovascularization, the choroidal neovascular is made to regress even by a single dose, and high therapeutic efficacy can be achieved.

### Pharmaceutical Composition

The pharmaceutical composition which includes the conjugate of the anti-VEGFR2 antibody and the photosensitizer can be prepared in a dosage form appropriate to various administration routes exemplified above. Dosage forms of the pharmaceutical composition of the present invention, in addition to injectable drugs and drops appropriate for intravitreal administration, intravenous administration, and intraperitoneal administration, include dosage forms for topical ocular administration such as eye drops and eye ointment, solid dosage forms such as tablets, granules, powdered drug, encapsulated formulations, and chewing agents, liquid dosage forms such as liquid drugs, syrups, and drops, adhesive skin patches, gels, and intercalating agents. The pharmaceutical composition of the present invention is preferably an aqueous liquid dosage form. Such pharmaceutical composition can be prepared by using conventional techniques commonly used in the field.

An amount (dosage) of the conjugate of the anti-VEGFR2 antibody and the photosensitizer in the pharmaceutical composition of the conjugate of the anti-VEGFR2 antibody and the photosensitizer may be an amount which is adequate for achieving the desired effect, and can be selected appropriately upon taking into consideration factors such as the subject to be treated, the lesion, the administration route, and the frequency of administration. As an example, the dosage (therapeutically effective amount) of the conjugate of the anti-VEGFR2 antibody and the photosensitizer in the pharmaceutical composition of the present invention, based on weight, is at least 0.1 g/kg, preferably at least 1 g/kg, and more preferably at least 10 g/kg, and moreover, is preferably 50 mg/kg or less, and more preferably 10 mg/kg or less, and for example, is in a range of 0.1 g/kg to 50 mg/kg, and preferably in a range of 1 g/kg to 10 mg/kg.

In a case of the intravenous administration, the dosage (therapeutically effective amount) of the conjugate of the anti-VEGFR2 antibody and the photosensitizer in the pharmaceutical composition of the present invention is preferably 0.1 mg/kg or more, and more preferably 1 mg/kg or more, and moreover, is preferably 50 mg/kg or less, and more preferably 10 mg/kg or less, and for example, can be made to be approximately 5 mg/kg, and for example, can be made to be in a range of 0.1 mg/kg to 50 mg/kg, preferably in a range of 1 mg/kg to 10 mg/kg, and more preferably in a range of 1 mg/kg to 5 mg/kg.

In a case of the intravitreal administration, the dosage (therapeutically effective amount) of the conjugate of the anti-VEGFR2 antibody and the photosensitizer in the pharmaceutical composition of the present invention, a single dosage for one eye is preferably 0.01 mg or more per dose and more preferably 0.1 mg or more per dose, and moreover, is preferably 10 mg or less per dose, more preferably 1.5 mg or less per dose, and for example, can be made to be approximately 0.625 mg per dose. The dosage (therapeutically effective amount), for example, can be made to be in a range of 0.01 mg per dose to 10 mg per dose, and preferably in a range of 0.1 mg per dose to 1.5 mg per dose. In a case in which the subject to be administered is a human, the dosage (therapeutically effective amount) is preferably in a range of 0.5 mg per dose to 1.5 mg per dose, and more preferably in a range of 0.625 mg per dose to 1.25 mg per dose.

In addition to the conjugate of the anti-VEGFR2 antibody and the photosensitizer, an appropriate amount of other component, within a scope that does not impair the effect of the present invention can be blended in the pharmaceutical composition which includes the conjugate of the anti-VEGFR2 antibody and the photosensitizer of the present invention. Examples of other components include arbitrary carriers, isotonic/isosmotic agents, buffering agents, pH adjusters, dilution agents, excipients, suspending agents, lubricating agents, glidants, adjuvants, media, delivery systems, emulsifiers, amino acids, saccharides, tablet disintegrants, absorbents, preserving agents, stabilizers, thickening agents, surfactants, bases, components for DDS, encapsulation materials, coating materials, coloring agents, aroma chemicals, or sweeteners. These components can be blended singly or upon appropriately combining two or more types thereof.

As an example, the pharmaceutical composition of the present invention for intravitreal administration or intravenous administration may include in addition to the aqueous solvent (such as water, physiological saline, phosphate buffered saline), one or more of an isotonic/isosmotic agent, a pH adjuster, an emulsifier, and a component for solubility and preventing aggregation of peptides/proteins such as a non-hydrophobic amino acid, a polyol, a saccharide, an osmolyte, and a nonionic surfactant.

### <Irradiation of Red Visible Light Rays or Near-Infrared Light Rays>

The pharmaceutical composition which includes the conjugate of the anti-VEGFR2 antibody and the photosensitizer is administered and the conjugate of the anti-VEGFR2 antibody and the photosensitizer is attached to the VEGFR2 which is highly expressing in the lesions. By irradiating the red visible light rays or the near-infrared light rays to the conjugate of the anti-VEGFR2 antibody and the photosensitizer attached to the lesions, damage is caused to neovascular lesions by photosensitizing effect of the photosensitizer in the conjugate, and the neovascular vessel is made to contract and regress.

### Wavelength

The wavelength region of the red visible light rays or the near-infrared light rays is from 650 nm to 900 nm, preferably from 660 nm to 850 nm, and more preferably 670 nm to 750 nm, for the reason such as the light rays in this wavelength region can easily reach a deep portion of a living body, have a small adverse effect on a living body, and a proportion thereof absorbed in a light absorbing material in the living body is small.

### Irradiation

An attachment of the conjugate of the anti-VEGFR2 antibody and the photosensitizer or the pharmaceutical composition including the conjugate not being specific to the lesions of age-related macular degeneration, the conjugate of the anti-VEGFR2 antibody and the photosensitizer exists also at sites other than lesions of the age-related macular degeneration. Therefore, it is preferable to irradiate the red visible light rays or the near-infrared light rays only to the lesions of the age-related macular degeneration.

An irradiation exposure dose can be set appropriately according to the conditions of the lesions, and in general, is 0.1 J/cm² or more, preferably 1 J/cm² or more, and more preferably 10 J/cm² or more, and moreover, is 500 J/cm² or less, preferably 100 J/cm² or less, and more preferably 75 J/cm² or less, and even more preferably 60 J/cm² or less, and further more preferably 50 J/cm² or less, and all the more preferably 30 J/cm² or less, and from a viewpoint of reducing the burden on the subject, is preferably in a range of 1 J/cm² to 75 J/cm², more preferably in a range of 1 J/cm² to 60 J/cm², even more preferably in a range of 1 J/cm² to 50 J/cm², and further more preferably in a range of 1 J/cm² to 30 J/cm² (for example, approximately 10 J/cm²). The number of irradiations may be one, or two or more than two.

The irradiation output can be set appropriately according to the condition of the lesions, and in general, from a viewpoint of reducing the burden on the subject, is preferably in a range of 100 mW/cm² to 1000 mW/cm², more preferably in a range of 200 mW/cm² to 800 mW/cm², and even more preferably in a range of 250 mW/cm² to 650 mW/cm².

The irradiation time can be set appropriately according to the condition of the lesions, and in general, from a viewpoint of reducing the burden on the subject, is preferably in a range of 40 seconds to 180 seconds, more preferably in a range of 80 seconds to 160 seconds, and even more preferably in a range of 90 seconds to 130 seconds.

In one embodiment, by combining a laser irradiation apparatus and a laser slit-lamp microscope used in an ophthalmic treatment, it is possible to irradiate laser while observing the lesion site, and it is possible to carry out the minimum necessary spot-laser irradiation avoiding normal tissues.

An area irradiated by the spot-laser irradiation can be made to be preferably 200% or less of an area of the lesion site (site to be irradiated), more preferably 150% or less of the area of the lesion site, and even more preferably 120% or less of the area of the lesion site. An irradiation diameter of the spot-laser radiation is preferably in a range of 50 m to 10000 μm, more preferably in a range of 500 μm to 5000 μm, and even more preferably in a range of 1000 μm to 1500 μm.

Moreover, in one embodiment, it is more preferable to combine the dosage of the photosensitizer and a laser irradiation intensity in an appropriate range. For instance, when the dosage of the conjugate of the anti-VEGFR2 antibody and the photosensitizer is in a range of 5 mg per dose to 10 mg per dose (intravenous administration), or in a range of 0.625 mg per dose to 1.25 mg per dose (intravitreal administration), it is preferable to make the amount of irradiation exposure to be in a range of 1 J/cm² to 50 J/cm². By combining the dosage and the intensity of irradiation in such manner, it is possible to suppress side effects of drug administration and side effects of laser irradiation to the minimum.

Moreover, in one embodiment, from a viewpoint of localizing the photosensitizer (conjugate of the anti-VEGFR2 antibody and the photosensitizer) to the targeted site while reducing the burden on the subject, it is preferable to make the time interval from the administration of the photosensitizer (conjugate of the anti-VEGFR2 antibody and the photosensitizer) up to the laser irradiation to be 168 hours or less, more preferably 120 hours or less, even more preferably 100 hours or less, and further more preferably 50 hours or less, and moreover, preferably to be 10 minutes or more, more preferably one hour or more, even more preferably 10 hours or more, and further more preferably 20 hours or more. In other words, a step of irradiating the red visible light rays or the near-infrared light rays to an eye of the subject is preferably carried out after a time in a range of 10 minutes to 168 hours after administering the conjugate of anti-VEGFR2 antibody and the photosensitizer, and more preferably after a time in a range of one hour to 120 hours after administering the conjugate, even more preferably after a time in a range of 10 hours to 100 hours after administering the conjugate, and further more preferably after a time in a range of 20 hours to 50 hours after administering the conjugate.

### <Subject>

The target of treatment for age-related macular degeneration by using the conjugate of the anti-VEGFR2 antibody and the photosensitizer or the pharmaceutical composition including the same of the present invention may be mammals including human, and non-human mammals including mice, dogs, cats, cows, and horses, but is preferably humans.

### <Target Diseases>

The conjugate of the anti-VEGFR2 antibody and the photosensitizer or the pharmaceutical composition including the same of the present invention is effective in the treatment of exudative age-related macular degeneration. The method for treating by using the anti-VEGFR2 antibody and the photosensitizer and the pharmaceutical composition using the same of the present invention may be effective at any of the stages from an initial stage to an advanced stage of the age-related macular degeneration. Moreover, the method for treating by using the anti-VEGFR2 antibody and the photosensitizer or the pharmaceutical composition using the same of the present invention may be effective for a disease involving pathological proliferation and growth due to VEGF such as retinopathy of prematurity, diabetic retinopathy, neovascular glaucoma, myopic choroidal neovascularization, and retinal vein occlusion involving proliferation of neovascular.

Although preferred embodiments of the present invention are described in the present specification, it is obvious to one skilled in the art that such embodiments are provided with the purpose of exemplifying only, and it is possible to add various variations, modifications, and substitutions without departing from the scope of the invention. It should be understood that various alternative embodiments of the invention described in the present specification may be used for carrying out the present invention. Moreover, the content described in all publications including patents and patent application documents referred to in the present specification should be construed to be incorporated in the same manner as the content explicitly stated in the present specification by the citation thereof.

### EXAMPLES

### Example 1

A total of four groups (n=8 for each group) of seven-week-old mice (C57BL6/J, CLEA Japan Inc.) were anesthetized by injecting a mixture of three anesthetics (domitor+midazolam+Vetorphale (0.75 mg/kg+4 mg/kg+5 mg/kg)) intraperitoneally, and pupils were dilated by using 5% tropicamide and 5% phenylephrine hydrochloride. For each eye, laser-spot irradiation (170 mW, 75 μm, 100 ms) was carried out at three to five locations surrounding an optic nerve, and CNV (choroidal neovascularization) model mice were prepared. Rupture of Bruch's membrane was verified by air bubbles generated at the time of laser irradiation. After a treatment of laser photocoagulation, the mice were awakened by administering anti-sedan and were returned to rearing cages.

Four days after the treatment of laser photocoagulation, 100 g of a DC101-IR700 conjugate was administered intraperitoneally to the mice of second group and the fourth group. PBS (phosphate buffered saline) was administered to the mice of the first group and the third group as a control sample. The DC101-IR700 conjugate was prepared by mixing a DC101 antibody and IR700 pigments under predetermined reaction conditions and removing the pigments that are not attached based on molecular weight.

Five days after the treatment of laser photocoagulation, the mice were anesthetized by injecting a mixture of three anesthetics (domitor+midazolam+Vetorphale (0.75 mg/kg+4 mg/kg+5 mg/kg)) intraperitoneally, and pupils were dilated by using 5% tropicamide and 5% phenylephrine hydrochloride. 12 J/cm² of red visible light rays or near-infrared light rays (wavelength: 690 nm) was irradiated (for 120 seconds at irradiation output of 10 mW/cm²) to one eye at a time of the mice of the third group and the fourth group. The red visible light rays or the near-infrared light rays were not irradiated to the mice of the first group and the second group. After irradiating the red visible light rays or the near-infrared light rays, the mice were awakened by administering anti-sedan and were returned to the rearing cages.

The mice were euthanized 10 days after the treatment of laser photocoagulation, and ocular globes were enucleated and fixed for one hour in 4% paraformaldehyde. After removing an anterior eye part and a neural retina, radial incision was performed and a compound body of retinal pigment epithelium and choroid coat was flattened, and choroidal flat-mount was prepared. After incubating for 15 minutes with PBS containing 0.05% Triton (registered trademark) X-100, it was incubated with 5% BSA (bovine serum albumin). Thereafter, it was incubated overnight with fluorescently-labelled isolectin-B4 at 4° C. The choroidal flat-mount was observed with a confocal laser microscope (SP5, Leica), and a volume of CNV was measured by an image analysis software IMARIS (Oxford Instruments) from images captured.

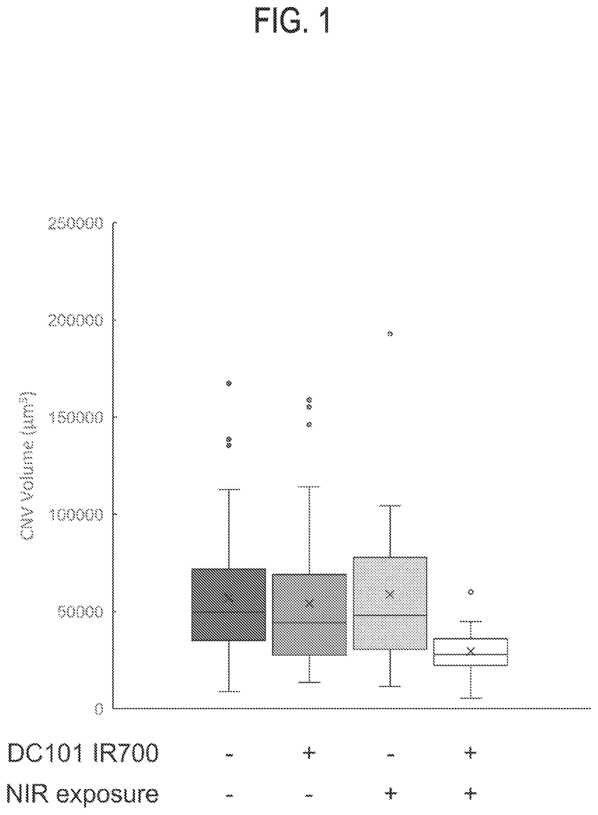

Results of measurement of the volume of CNV are shown in FIG. 1. A vertical axis indicates the volume of CNV. No significant changes were observed between an untreated group, the second group subjected to DC101-IR700 administration alone, and the third group subjected to irradiation of red visible light rays or near-infrared light rays alone. A significant decrease in the volume of CNV as compared to that in the other groups was observed for the fourth group to which the DC101-IR700 was administered as well as the red visible light rays or the near-infrared light rays were irradiated.

### Example 2

A total of three groups (n=6 for each group) of seven-week-old mice (C57BL6/J, CLEA Japan Inc.) were anesthetized by injecting a mixture of three anesthetics (domitor+midazolam+Vetorphale (0.75 mg/kg+4 mg/kg+5 mg/kg)) intraperitoneally, and pupils were dilated by using 5% tropicamide and 5% phenylephrine hydrochloride. For each eye, laser-spot irradiation (170 mW, 75 μm, 100 ms) was carried out at three locations surrounding an optic nerve, and CNV (choroidal neovascularization) model mice were prepared. Rupture of Bruch's membrane was verified by air bubbles generated at the time of laser irradiation. After a treatment of laser photocoagulation, the mice were awakened by administering anti-sedan and were returned to rearing cages.

Five days after the treatment of laser photocoagulation, 100 g of a DC101-IR700 conjugate was administered intraperitoneally to the mice of the second group and the third group. PBS was administered to the first group as a control sample. Six days after the treatment of laser photocoagulation, the mice were anesthetized by injecting a mixture of three anesthetics (domitor+midazolam+Vetorphale (0.75 mg/kg, 4 mg/kg, 5 mg/kg)) intraperitoneally, and pupils were dilated by using 5% tropicamide and 5% phenylephrine hydrochloride. 100 J/cm² of red visible light rays or near-infrared light rays was irradiated (for 166 seconds at irradiation output of 600 mW/cm²) to one eye at a time of the mice of the third group by using VISULAS PDT (photodynamic therapy) system 609S while observing with a slit-lamp microscope. The irradiation diameter of the spot laser irradiation was 1200 μm. The red visible light rays or the near-infrared light rays were not irradiated to the mice of the first group and the second group. After laser irradiation, the mice were awakened by administering anti-sedan and were returned to the rearing cages.

The mice were euthanized 10 days after the treatment of laser photocoagulation, and ocular globes were enucleated and fixed for one hour in 4% paraformaldehyde. After removing an anterior eye part and a neural retina, radial incision was performed and a compound body of retinal pigment epithelium and choroid coat was flattened, and a choroidal flat-mount was prepared. After incubation for 15 minutes with PBS containing 0.05% Triton (registered trademark) X-100, it was incubated with 5% BSA. Thereafter, it was incubated overnight with fluorescently-labelled isolectin-B4 at 4° C. The choroidal flat-mount was observed with a confocal laser microscope (SP5, Leica), and a volume of CNV was measured by an image analysis software IMARIS (Oxford Instruments) from images captured.

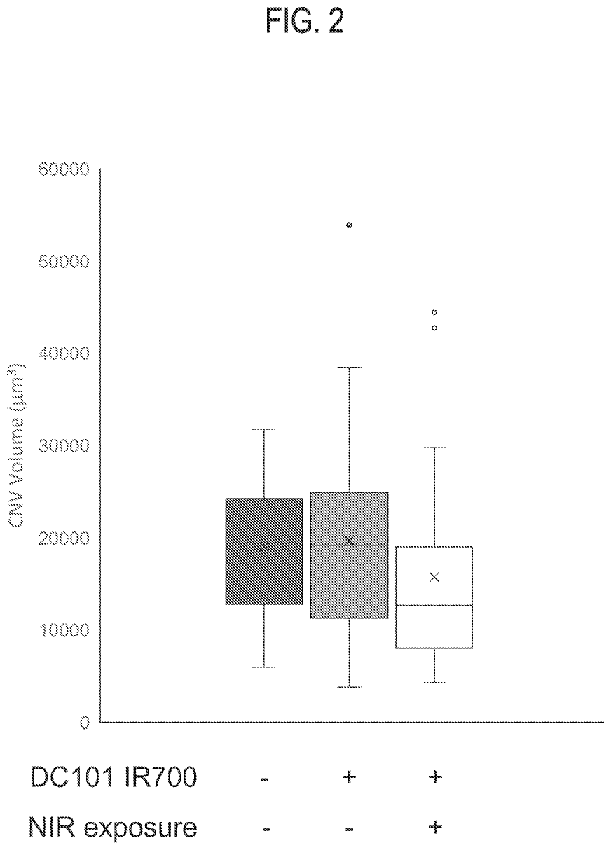

Results of measurement of the volume are shown in FIG. 2. A vertical axis indicates the volume of CNV. No significant changes were observed between an untreated group (first group) and the second group subjected to DC101-IR700 administration alone. A tendency for decrease in the volume of CNV as compared to that for the other groups was observed for the third group to which DC1101-IR700 was administered as well as the red visible light rays or the near-infrared light rays were irradiated.

### Example 3

A total of three groups (n=6 for each group) of seven-week-old mice (C57BL6/J, CLEA Japan Inc.) were anesthetized by injecting a mixture of three anesthetics (domitor+midazolam+Vetorphale (0.75 mg/kg+4 mg/kg+5 mg/kg)) intraperitoneally, and pupils were dilated by using 5% tropicamide and 5% phenylephrine hydrochloride. For each eye, laser-spot irradiation (170 mW, 75 μm, 100 ms) was carried out at three locations surrounding an optic nerve, and CNV (choroidal neovascularization) model mice were prepared. Rupture of Bruch's membrane was verified by air bubbles generated at the time of laser irradiation. After a treatment of laser photocoagulation, the mice were awakened by administering anti-sedan and were returned to rearing cages.

Six days after the treatment of laser photocoagulation, the mice were anesthetized by injecting a mixture of three anesthetics (domitor+midazolam+Vetorphale (0.75 mg/kg+4 mg/kg+5 mg/kg)) and 600 ng of a DC101-IR700 conjugate was administered intravitreally to the mice of the second group and the third group. PBS was administered to the first group as a control sample. The mice were awakened by administering anti-sedan and were returned to the rearing cages.

Seven days after the treatment of laser photocoagulation, the mice were anesthetized by injecting the mixture of three anesthetics intraperitoneally, and pupils were dilated by using 5% tropicamide and 5% phenylephrine hydrochloride. 12 J/cm² of red visible light rays or near-infrared light rays was irradiated (for 120 seconds at irradiation output 10 mW/cm²) to one eye at a time of the mice. After irradiating the red visible light rays or the near-infrared light rays, the mice were awakened by administering anti-sedan and were returned to the rearing cages.

The mice were euthanized 10 days after the treatment of laser photocoagulation, and ocular globes were enucleated and fixed for one hour in 4% paraformaldehyde. After removing an anterior eye part and a neural retina, radial incision was performed and a compound body of retinal pigment epithelium and choroid coat was flattened, and a choroidal flat-mount was prepared. After incubating for 15 minutes with PBS containing 0.05% Triton (registered trademark) X-100, it was incubated with 5% BSA. Thereafter, it was incubated overnight with fluorescently-labelled isolectin-B4 at 4° C. The choroidal flat-mount was observed with a confocal laser microscope (SP5, Leica), and a volume of CNV was measured by an image analysis software IMARIS (Oxford Instruments) from images captured.

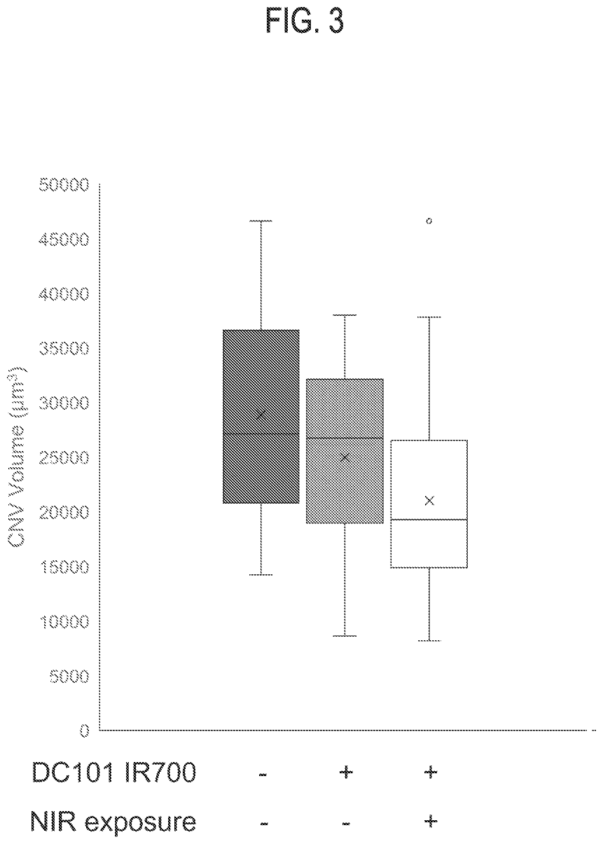

Results of measurement of the volume are shown in FIG. 3. A vertical axis indicates the volume of CNV. No significant changes were observed between the second group subjected to DC101-IR700 administration alone. A tendency for decrease in the volume of CNV as compared to that for the other groups was observed for the third group to which DC101-IR700 was administered as well as the red visible light rays or the near-infrared light rays were irradiated.

From these results, it was verified that by photoimmunotherapy in which the anti-VEGFR2 antibody and the photosensitizer are used, the choroidal neovascularization which is the essential of the exudated age-related macular degeneration can be regressed. This indicates that photoimmunotherapy in which the anti-VEGFR2 antibody and the photosensitizer are used can provide a curative treatment for the exudative age-related macular degeneration, and such results were the findings first discovered by the inventors of the present invention.

### Example 4 Protocols Envisaged for Humans

1.25 mg of a conjugate of an anti-VEGFR2 antibody and a photosensitizer is to be administered locally by an intravitreal injection, and 50 J/cm² of red visible light rays or near-infrared light rays is to be irradiated to a lesion site by using a slit-lamp microscope laser irradiation apparatus 24 hours after the intravitreal injection.

### Example 5

A total of two groups (n==5 for each group) of seven-week-old mice (C57BL6/J, CLEA Japan Inc.) were anesthetized by injecting a mixture of three anesthetics (domitor+midazolam+Vetorphale (0.75 mg/kg+4 mg/kg+5 mg/kg)) intraperitoneally, and pupils were dilated by using 5% tropicamide and 5% phenylephrine hydrochloride. For each eye, laser-spot irradiation (170 mW, 75 μm, 100 ms) was carried out at three to five locations surrounding an optic nerve, and CNV (choroidal neovascularization) model mice were prepared. Rupture of Bruch's membrane was verified by air bubbles generated at the time of laser irradiation. After a treatment of laser photocoagulation, the mice were awakened by administering anti-sedan and were returned to rearing cages.

Six days after the treatment of laser photocoagulation, 100 g of a DC101-Alexa488 conjugate was administered intraperitoneally to the mice of the first group. PBS (phosphate buffered saline) was administered to the second group as a control sample. The DC101-Alexa488 conjugate was prepared by mixing a DC101 antibody and Alexa488 pigments under predetermined reaction conditions and removing the pigments that are not attached based on molecular weight.

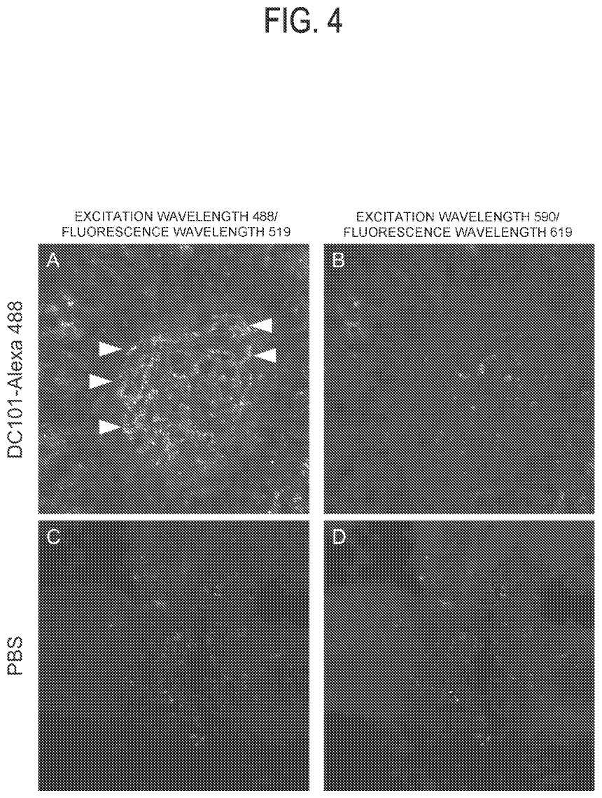

The mice were euthanized seven days after the treatment of laser photocoagulation, which is 24 hours after the administration of the DC101-Alexa488 conjugate, and ocular globes were enucleated and fixed for one hour in 4% paraformaldehyde. After removing an anterior part and a neural retina, radial incision was performed and a compound body of retina pigment epithelium and choroid coat was flattened, and a choroidal flat-mount was prepared. The choroidal flat-mount was observed with a confocal laser microscope (SP5, Leica), and images thereof were captured. Results are shown in FIG. 4.

As shown in FIG. 4, at an excitation wavelength of 488 nm for the group subjected to the DC101-Alex488 administration, a vascular signal in the CNV was observed (A). This signal was not consistent with a signal by autofluorescence observed at the excitation wavelength of 590 nm (B). A vascular structure was not observed at the excitation wavelength of 488 nm in the group subjected to PBS administration, and only a signal consistent with the autofluorescence was observed (C, D). From these observations, it was revealed that the conjugate of the anti-VEGFR2 antibody and the photosensitizer is localized at the target site even 24 hours after the administration of the conjugate of the anti-VEGFR2 antibody and the photosensitizer.

1. A method for treating exudative age-related macular degeneration for a subject requiring treatment, comprising:
administering a conjugate of an anti-VEGFR2 antibody and a photosensitizer to the subject; and
irradiating red visible light rays or near-infrared light rays to an eye of the subject.

2. The method according to claim 1, wherein the conjugate of the anti-VEGFR2 antibody and the photosensitizer is administered intravitreally.

3. The method according to claim 1, wherein the conjugate of the anti-VEGFR2 antibody and the photosensitizer is administered intravenously.

4. The method according to claim 1, wherein a wavelength of the red visible light rays or the near-infrared light rays is in a range of 650 nm to 900 nm.

5. The method according to claim 1, wherein an exposure irradiation dose of the red visible light rays or the near-infrared light rays is in a range of 1 J/cm² to 75 J/cm².

6. The method according to claim 1, wherein an irradiation output of the red visible light rays or the near-infrared light rays is in a range of 100 mW/cm² to 1000 mW/cm².

7. The method according to claim 1, wherein an irradiation time of the red visible light rays or the near-infrared light rays is in a range of 40 seconds to 180 seconds.

8. The method according to claim 1, wherein the photosensitizer is IR700.

9. The method according to claim 1, wherein irradiating the red visible light rays or the near-infrared light rays to the eye of the subject includes irradiating the red visible light rays or the near-infrared light rays by a laser irradiating apparatus while observing a lesion site by using a slit-lamp microscope.

10. The method according to claim 1, wherein irradiating the red visible light rays or the near-infrared light rays to the eye of the subject is carried out after a time in a range of 10 minutes to 168 hours after administering the conjugate of the anti-VEGFR2 antibody and the photosensitizer to the subject.

11. A pharmaceutical composition for treating exudative age-related macular degeneration, comprising:
a therapeutically effective amount of a conjugate of an anti-VEGFR2 antibody and a photosensitizer

12. A use of a therapeutically effective amount of a conjugate of an anti-VEGFR2 antibody and a photosensitizer for manufacturing a pharmaceutical composition for treating exudative age-related macular degeneration.
